(19) United States
(12) Patent Application Publication
(10) Pub. No.: US 2014/0277182 A1
(43) Pub. Date: Sep. 18, 2014

(54) SPINAL IMPLANT SYSTEM AND METHOD

(71) Applicant: WARSAW ORTHOPEDIC, INC., Warsaw, IN (US)

(72) Inventors: Jeff R. Justis, Germantown, TN (US); Alison G. Powers, Memphis, TN (US); Gary S. Lindemann, Collierville, TN (US)

(73) Assignee: WARSAW ORTHOPEDIC, INC., Warsaw, IN (US)

(21) Appl. No.: 13/796,253

(22) Filed: Mar. 12, 2013

(51) Int. Cl. A61B 17/70

(52) U.S. Cl. CPC A61B 17/7059
USPC 606/293

(57) ABSTRACT

A spinal implant includes a wall defining a plane and having a first surface and a second surface configured to engage tissue. The wall defines a first opening configured for disposal of a first bone fastener and a second opening configured for disposal of a second bone fastener. The wall includes an inner surface that defines a passageway disposed between the first and second openings. A part configured for disposal in the passageway. The first bone fastener is engageable with the part such that the part translates relative to the inner surface in the plane to engage the second bone fastener and to resist backout of the second bone fastener from the second opening. Systems and methods are disclosed.

### TECHNICAL FIELD

The present disclosure generally relates to medical devices for the treatment of musculoskeletal disorders, and more particularly to a surgical system including an anterior spinal implant and a method for deformity correction.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. Correction treatments used for positioning and alignment may employ implants, such as vertebral rods, plates and fasteners, for stabilization of a treated section of a spine. This disclosure describes an improvement over these prior art technologies.

### SUMMARY

In one embodiment, in accordance with the principles of the present disclosure, a spinal implant is provided. The spinal implant comprises a wall defining a plane and having a first surface and a second surface configured to engage tissue. The wall defines a first opening configured for disposal of a first bone fastener and a second opening configured for disposal of a second bone fastener. The wall includes an inner surface that defines a passageway disposed between the first and second openings. A part is configured for disposal in the passageway. The first bone fastener is engageable with the part such that the part translates relative to the inner surface in the plane to engage the second bone fastener and to resist backout of the second bone fastener from the second opening. In some embodiments, systems and methods are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a system in accordance with the principles of the present disclosure;

FIG. 2 is a cross section view of the components shown in FIG. 1 taken along line II-II;

FIG. 3 is a plan view of the components shown in FIG. 1;

FIG. 4 is a plan view of the components shown in FIG. 1;

FIG. 5 is a perspective view of the components of one embodiment of a system in accordance with the principles of the present disclosure;

FIG. 6 is a plan view of components of one embodiment of a system in accordance with the principles of the present disclosure;

FIG. 7 is a perspective view of components of one embodiment of a system in accordance with the principles of the present disclosure with parts separated;

FIG. 8 is a plan view of the components shown in FIG. 7; and

FIG. 9 is a perspective view of components of one embodiment of a system in accordance with the principles of the present disclosure.

### DETAILED DESCRIPTION

The exemplary embodiments of the spinal implant system and method are discussed in terms of medical devices for the treatment of musculoskeletal disorders and more particularly, in terms of a surgical system and method for treatment of a spine disorder. In some embodiments, the spinal implant system and method may be employed in applications such as correction of deformities, such as, for example, scoliosis. In some embodiments, for example, the spinal implant system and method can include attachment of a tether to a first side, such as, for example, a convex side of a spine that is curved due to scoliosis. In some embodiments, the tether may be affixed to a first side of each of a plurality of vertebrae to prevent growth of vertebrae of the first side, and the system allows for growth and adjustments to a second side, such as, for example, a concave side of the plurality of vertebrae. In one embodiment, the spinal implant system and method provides for resistance to and/or prevention of the backing out of a bone fastener, for example, a screw, from an opening of the spinal implant caused by the growth and adjustments of the plurality of vertebrae.

In one embodiment, the system includes a spinal implant that locks a bone fastener, such as, for example, a bone screw with a plate to prevent the backing out of the bone screw from the plate. In one embodiment, the spinal implant includes an integrated slider lock and a plate. In one embodiment, the plate includes an opening configured for disposal of an anchoring bone screw and an opening configured for disposal of a larger bone screw. In one embodiment, the anchoring bone screw is disposed within a first opening and the larger screw is inserted into a second opening. In one embodiment, the larger screw engages a slider lock causing the slider lock to translate within the plate until the slider lock is covering a shelf of the anchoring bone screw, inhibiting backout of the anchoring bone screw from the plate.

In one embodiment, the spinal implant includes an integrated plate locking mechanism. In one embodiment, the integrated plate locking mechanism includes a cam lock that is keyed and retained within a plate. In one embodiment, the system includes a first anchoring bone screw configured for disposal in a first opening of the plate and a second larger bone screw configured for disposal in a second opening of the plate. In one embodiment, the first screw is inserted in one of the openings in the plate and the larger screw is inserted into the other opening and engages the cam lock causing the cam lock to be pushed over to cover a shelf of the first bone screw. This configuration inhibits back out of the first bone screw from the plate. In one embodiment, the plate is used anteriorly as part of a fusion-less tether system.

In some embodiments, one or all of the components of the spinal implant system may be disposable, peel-pack, pre-packed sterile devices. One or all of the components of the spinal implant system may be reusable. In some embodiments, the spinal implant system may be configured as a kit with multiple sized and configured components.

In some embodiments, the present disclosure may be employed to treat spinal disorders, such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed spinal implant system and method may be employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The present disclosure may also be employed with procedures for treating the lumbar, cervical, thoracic, sacral and pelvic regions of a spinal column. The present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

The following discussion includes a description of a spinal implant system, related components and methods for employing the spinal implant system. Alternate embodiments are also disclosed. Reference will now be made in detail to the exemplary embodiments of the present disclosure, which are illustrated in the accompanying figures. Turning to FIGS. 1-5, there are illustrated components of a spinal implant system 10, in accordance with the principles of the present disclosure.

The components of system 10 can be fabricated from biologically acceptable materials suitable for medical applications, including metals, synthetic polymers, ceramics and bone material and/or their composites, depending on the particular application and/or preference of a medical practitioner. For example, the components of system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, super elastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyimide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of system 10 may have material composites, including the above materials, to achieve various desired characteristics such as strength, rigidity, elasticity, compliance, biomechanical performance, durability and radiolucency or imaging preference. The components of system 10, individually or collectively, may also be fabricated from a heterogeneous material such as a combination of two or more of the above-described materials. The components of system 10 may be monolithically formed, integrally connected or include fastening elements and/or instruments, as described herein.

System 10 is employed, for example, with an open, mini-open or minimally invasive surgical technique, which may include percutaneous, to attach a longitudinal element, such as, for example, a tether, to a first side, such as, for example, a convex side of a spine that has a spinal disorder. The tether may be attached to a bone fastener, which is disposed with an implant, such as, for example, a plate, affixed to the convex side of each of a plurality of vertebrae to prevent growth of vertebrae of a selected section of the spine. System 10 allows for growth and adjustments to a second side, such as, for example, a concave side of the plurality of vertebrae for a correction treatment to treat various spine pathologies, such as, for example, adolescent idiopathic scoliosis and Scheuermann's kyphosis.

System 10 includes a spinal implant, such as, for example, a plate 12 having a substantially rectangular configuration. In some embodiments, plate 12 can be variously configured, such as, for example, tubular, oval, oblong, triangular, square, polygonal, irregular, uniform, non-uniform, variable, hollow and/or tapered. Plate 12 includes a wall 14 extending between an end 16 and an end 18. Wall 14 defines and is disposed in at least a portion of a plane P1. Wall 14 has a substantially rectangular cross section and defines a thickness to accommodate components of system 10. In some embodiments, wall 14 can have alternate cross-section and/or thickness configurations, such as, for example, arcuate, undulating, offset, staggered, tubular, oval, oblong, triangular, square, polygonal, irregular, uniform, non-uniform, variable, hollow and/or tapered.

Wall 14 includes a surface 20 and a surface 22. Surface 22 includes substantially planar portions and is oriented in a first direction such that all or only a portion of surface 22 faces and/or engages tissue, as will be described. Surface 20 is oriented in a second direction, opposite to the first direction. In one embodiment, surface 22 is oriented in a posterior or postero-lateral direction for engagement with vertebral tissue. In one embodiment, surface 22 has a frictional surface configuration for engagement with tissue to enhance fixation. In some embodiments, surface 22 may include alternate surface configurations, such as, for example, rough, arcuate, undulating, mesh, porous, semi-porous, dimpled and/or textured according to the requirements of a particular application.

Plate 12 has a double plate configuration such that wall 14 includes an opening 24 and an opening 26 spaced apart from opening 24 along surface 20. Openings 24, 26 are substantially circular and extend through the thickness of wall 14. In some embodiments, opening 24 and/or opening 26 can be variously configured, such as, for example, oval, oblong, triangular, square, polygonal, irregular, uniform, non-uniform and/or tapered.

Opening 24 is configured to receive a bone fastener, such as, for example, a bone screw 28, as shown in FIG. 5, which connects a longitudinal element (not shown), such as, for example, a tether, to plate 12 and/or tissue, as will be described. Bone screw 28 has a length disposed along a longitudinal axis. Bone screw 28 comprises a proximal portion, such as, for example, a head 30. Head 30 defines an implant cavity 32. Bone screw 28 comprises a distal portion, such as, for example, an elongated shaft 34 configured for penetrating tissue.

Shaft 34 of bone screw 28 has a cylindrical cross section configuration and includes an outer surface having an external thread form. In some embodiments, the external thread form may include a single thread turn or a plurality of discrete threads. In some embodiments, other engaging structures may be located on shaft 34, such as, for example, a nail configuration, barbs, expanding elements, raised elements and/or spikes to facilitate engagement of shaft 34 with tissue, such as, for example, vertebrae.

In some embodiments, all or only a portion of shaft 34 may have alternate cross section configurations, such as, for example, oval, oblong, triangular, square, polygonal, irregular, uniform, non-uniform, offset, staggered, undulating, arcuate, variable and/or tapered. In some embodiments, the outer surface of shaft 34 may include one or a plurality of openings. In some embodiments, all or only a portion of the outer surface of shaft 34 may have alternate surface configurations to enhance fixation with tissue such as, for example, rough, arcuate, undulating, mesh, porous, semi-porous, dimpled and/or textured according to the requirements of a particular application. In some embodiments, all or only a portion of shaft 34 may be disposed at alternate orientations, relative to the longitudinal axis, such as, for example, transverse, perpendicular and/or other angular orientations such as acute or obtuse, co-axial and/or may be offset or staggered. In some embodiments, all or only a portion of shaft 34 may be cannulated.

Head 30 includes a pair of spaced apart arms having an inner surface that defines a U-shaped passageway 36. Passageway 36 is configured for disposal of an implant, such as, for example, a tether. In some embodiments, all or only a portion of passageway 36 may have alternate cross section configurations, such as, for example, oval, oblong, triangular, square, polygonal, irregular, uniform, non-uniform, offset, staggered, and/or tapered. In some embodiments, the arms of head 30 may be disposed at alternate orientations, relative to the longitudinal axis, such as, for example, transverse, perpendicular and/or other angular orientations such as acute or obtuse, co-axial and/or may be offset or staggered.

The inner surface of head 30 includes a thread form configured for engagement with a coupling member, such as, for example, a set screw (not shown). Set screw is threadedly engaged with head 30 to attach, fix and/or lock the tether with bone screw 28 and/or plate 12, as described.

Opening 26 is configured to receive a bone fastener, such as, for example, a bone screw 38. Bone screw 38 connects plate 12 with tissue, as will be described. Bone screw 38 extends between a proximal portion, such as, for example, a head 40 and a distal portion, such as, for example, an elongated shaft 42. Shaft 42 is configured for penetrating vertebral tissue, similar to shaft 34, described above. Bone screw 38 includes an intermediate portion, such as, for example, a shoulder 43 disposed between shaft 42 and head 40. Shoulder 43 extends radially from bone screw 38.

Head 40 includes an inner surface that defines a socket cavity 46 configured for engagement with a tool or instrument (not shown) for inserting and tensioning bone screw 38 with tissue and/or plate 12. The inner surface that defines cavity 46 receives a surface of a drive element of a tool that matingly engages the inner surface for manipulating bone screw 38. In some embodiments, the surface of cavity 46 and the drive element can be alternatively configured, such as, for example, thread form, triangular, square, polygonal, hexalobular, star, torx, irregular, uniform, non-uniform, offset, staggered and/or tapered.

In some embodiments, system 10 can include one or a plurality of bone fasteners such as those described herein and/or fixation elements, which may be employed with a single vertebral level or a plurality of vertebral levels. In some embodiments, the bone fasteners and/or fixation elements may be engaged with vertebrae in various orientations, such as, for example, series, parallel, offset, staggered and/or alternate vertebral levels. In some embodiments, the bone fasteners may include one or a plurality of multi-axial screws, sagittal angulation screws, pedicle screws, mono-axial screws, uni-planar screws, fixed screws, tissue penetrating screws, conventional screws, expanding screws, anchors, buttons, connectors, clips, snaps, friction fittings, compressive fittings, expanding rivets, staples, nails, adhesives, fixation plates and/or posts. The bone fasteners and/or fixation elements may be coated with an osteoinductive or osteoconductive material to enhance fixation, and/or include one or a plurality of therapeutic agents.

Plate 12 includes an inner surface 48 that defines a passageway 50. Passageway 50 is disposed between openings 24, 26. In one embodiment, passageway 50 may extend between surfaces 20, 22 through a portion of the thickness of plate 12. Passageway 50 defines a cross-section to accommodate components of system 10. In some embodiments, passageway 50 can have alternate configurations and/or cross-section configurations, such as, for example, arcuate, undulating, offset, staggered, tubular, oval, oblong, triangular, square, polygonal, irregular, uniform, non-uniform, variable and/or tapered.

Wall 14 includes a flange 52 and a flange 54 extending in a cantilever configuration from inner surface 48 into passageway 50. Flange 52 is oriented in a first direction and flange 54 is oriented in a second direction, opposite to the first direction. Flanges 52, 54 are each substantially rectangular. In some embodiments, flange 52 and/or flange 54 can be variously configured, such as, for example, those alternatives described herein.

Flange 52 defines a channel 56 with inner surface 48 and flange 58 defines a channel 58 with inner surface 48. Channels 56, 58 extend between openings 24, 26. Channels 56, 58 are substantially rectangular. Channels 56, 58 are configured for disposal of a part of plate 12, described herein. Flange 52, flange 58 and/or inner surface 48, which defines channels 56, 58, are configured for slidable engagement with the part, for axial translation of the part relative to wall 14 along passageway 50 between a first orientation, as shown in FIG. 3, to overlap at least a portion of opening 24 such that screw 38 is moveable within opening 26 and a second orientation, as shown in FIG. 4, to overlap at least a portion of opening 26 such that the part resists backout of screw 38 from opening 26, as described herein. In some embodiments, channels 56, 58 can be variously configured, such as, for example, those alternatives described herein.

System 10 includes a part, such as, for example, slider 60 configured for moveable disposal in passageway 50. Slider 60 has a substantially rectangular configuration. In some embodiments, slider 60 can be variously configured, such as, for example, tubular, oval, oblong, triangular, square, polygonal, irregular, uniform, non-uniform, variable, hollow and/or tapered. Slider 60 defines a thickness t1 that is substantially non-uniform to facilitate engage and/or translation along and with the components of plate 12. In some embodiments, slider 60 can have alternate cross-section and/or thickness configurations, such as, for example, arcuate, undulating, offset, staggered, tubular, oval, oblong, triangular, square, polygonal, irregular, uniform, variable, hollow and/or tapered.

Slider 60 includes a first portion 76, an intermediate portion 78 and a second portion 80. Portion 76 is configured for engagement and overlap with bone screws 28, 38. Portion 76 has a width dimension w1 that is greater than a width dimension w2 of passageway 50 such that portion 76 overlaps surface 20 of plate 12 to facilitate retention of slider 60 with wall 14.

Portion 76 extends between an end 62 and an end 64. Portion 76 includes an inclined surface at end 62, such as, for example, an inclined cam surface 66 and an arcuate surface 68. Surfaces 66, 68 are engageable with bone screw 28 such that slider 60 translates relative to inner surface 48 in plane P1. Portion 76 includes an inclined surface at end 64, such as, for example, an inclined cam surface 70 and an arcuate surface 72. Surfaces 70, 72 are engageable with bone screw 38. Surface 72 is dimensioned and shaped such that surface 72 is engaged with bone screw 38 in the second orientation, as described herein. In some embodiments, surfaces 66, 68, 70 and/or 72 may be variously shaped, such as, for example, planar, linear, undulating, offset, staggered, tubular, oval, oblong, triangular, square, polygonal, irregular, uniform, variable, hollow and/or tapered.

End 64 includes a substantially planar underside surface 74 configured to overlap and engage shoulder 43 of bone screw 38 in the second orientation to lock and/or fix screw 38 with plate 12. In some embodiments, underside surface 74 may have alternate surface configurations, such as, for example, those alternatives described herein.

Intermediate portion 78 extends between portion 76 and a portion 80. Intermediate portion 78 is configured for moveable disposal in passageway 50, which may include slidable engagement with inner surface 48. Intermediate portion 78 has a substantially square cross section configuration. In some embodiments, the cross section of intermediate portion 78 can be alternatively configured, such as, for example, those alternatives described herein.

Intermediate portion 78 defines a cavity 82 and a cavity 84. The surfaces of slider 60 that define cavity 82 define a female portion 83 configured to mate with flange 52 for retention of slider 60 with plate 12. Flange 52 is configured for moveable disposal with cavity 82 such that slider 60 translates relative to plate 12. The surfaces of slider 60 that define cavity 84 define a female portion 85 configured to mate with flange 54 for retention of slider 60 with plate 12. Flange 54 is configured for moveable disposal with cavity 84 such that slider 60 translates relative to plate 12. Cavities 82, 84 have a substantially square cross section configuration. In some embodiments, the cross section of cavities 82, 84 can be alternatively configured, such as, for example, those alternatives described herein.

Second portion 80 of slider lock 60 includes a flange 86 and a flange 88 that are engageable with flanges 52, 54, respectively, in a configuration to retain slider 60 with plate 12. Flange 86 is configured for moveable disposal in channel 56 of passageway 50 such that slider 60 translates relative to plate 12. Flange 88 is configured for moveable disposal in channel 58 of passageway 50 such that slider 60 translates relative to plate 12. Flanges 86, 88 have a substantially rectangular cross section configuration. In some embodiments, the cross section of flanges 86, 88 can be alternatively configured, such as, for example, those alternatives described herein.

In operation, plate 12 includes slider 60, which is oriented in an orientation such that end 62 overlaps a portion of opening 24, as shown in FIG. 3. In this orientation, bone screw 38 is moveable within opening 26. Bone screw 38 is axially translated into opening 26 and a drive tool is disposed with cavity 46 to rotate bone screw 38 into engagement with vertebral tissue.

Bone screw 28 is axially translated into opening 24. Head 30 of bone screw 28 engages surfaces 66, 68 of slider 60 causing slider 60 to translate, in the direction shown by arrow A in FIG. 3, relative to inner surface 48 in plane P1. Slider 60 translates in plane P1 such that surfaces 70, 72 engage bone screw 38. Slider 60 translates to an orientation, as shown in FIGS. 4 and 5, such that underside surface 74 of slider 60 overlaps a portion of opening 26 and engages shoulder 43 of bone screw 38. In this orientation, slider 60 engages shoulder 43 in a locking configuration of plate 12 with bone screw 38 to resist and/or prevent backout of bone screw 38 from opening 26.

Plate 12 includes a pair of spaced apart transverse extensions 90. Extensions 90 extend substantially perpendicular from surface 22 of plate 12 and are configured to penetrate tissue, such as, for example, bone. Each extension 90 includes fixation elements 92. In some embodiments, extensions 90 can have variously configured fixation elements, such as, for example, nails, serrated, textured, staggered, uneven, undulating, smooth, barbs and/or raised elements to facilitate engagement with tissue, such as, for example, a cortical wall of vertebrae.

System 10 includes a flexible longitudinal element, such as, for example, a flexible tether (not shown) configured for disposal in passageway 36. The tether has a flexible configuration, which includes movement in a lateral or side to side direction and prevents expanding and/or extension in an axial direction upon fixation with vertebrae. In some embodiments, all or only a portion of the tether may have a semi-rigid, rigid or elastic configuration, and/or have elastic properties, such as the elastic properties corresponding to the material examples described above, such that tether provides a selective amount of expansion and/or extension in an axial direction. In some embodiments, the tether may be compressible in an axial direction. In some embodiments, the tether can include a plurality of separately attachable or connectable portions or sections, such as bands or loops, or may be monolithically formed as a single continuous element. In one embodiment, system 10 includes two tethers. In some embodiments, the tether is configured to extend over one or a plurality of vertebral levels.

In some embodiments, the tether can have a uniform thickness/diameter. In some embodiments, the tether may have various surface configurations, such as, for example, rough, threaded for connection with surgical instruments, arcuate, undulating, porous, semi-porous, dimpled, polished and/or textured. In some embodiments, the thickness defined by the tether may be uniformly increasing or decreasing, or have alternate diameter dimensions along its length. In some embodiments, the tether may have various cross section configurations, such as, for example, oval, oblong, triangular, rectangular, square, polygonal, irregular, uniform, non-uniform, variable and/or tapered.

In some embodiments, the tether may have various lengths. In some embodiments, the tether may be braided, such as a rope, or include a plurality elongated elements to provide a predetermined force resistance. In some embodiments, the tether may be made from autograft and/or allograft, as described above, and be configured for resorbable or degradable applications. In one embodiment, the tether is a cadaver tendon. In some embodiments, the tether may include a cadaver ligament, solid core, tubular element, an artificial strand or a flexible rod.

In assembly, operation and use, spinal implant system 10, similar to the systems described herein, is employed with a surgical procedure, such as, for example, a correction treatment to treat adolescent idiopathic scoliosis and/or Scheuermann's kyphosis of a spine. In some embodiments, one or all of the components of spinal implant system 10 can be delivered or implanted as a pre-assembled device or can be assembled in situ. Spinal implant system 10 may be completely or partially revised, removed or replaced.

For example, as shown in FIG. 5, system 10 can be employed with a surgical correction treatment of an applicable condition or injury of an affected section of a spinal column and adjacent areas within a body (not shown), such as, for example, at least a first vertebra and a second vertebra. In some embodiments, spinal implant system 10 may be employed with one or a plurality of vertebrae.

In use, to treat a selected section of vertebrae, a medical practitioner obtains access to a surgical site including vertebrae in any appropriate manner, such as through incision and retraction of tissues. In some embodiments, spinal implant system 10 can be used in any existing surgical method or technique including open surgery, mini-open surgery, minimally invasive surgery and percutaneous surgical implantation, whereby vertebrae is accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for implantation of components of system 10. A preparation instrument (not shown) can be employed to prepare tissue surfaces of vertebrae, as well as for aspiration and irrigation of a surgical region according to the requirements of a particular surgical application.

Pilot holes are made in vertebrae for receiving bone screws 28, 38 that connect at least two plates 12 with vertebrae. Plates 12 are removably engaged with a delivery instrument (not shown) and delivered along the surgical pathway. Plates 12 are each delivered to a surgical site adjacent vertebrae. Plates 12 are each oriented for fixation with vertebrae.

With plates 12 disposed in a selected orientation relative to a selected vertebra, slider 60 is disposed in an orientation such that end 62 overlaps a portion of opening 24, as shown in FIG. 3. Bone screw 38 is axially translated into opening 26 and a drive tool (not shown) is disposed with cavity 46 to rotate bone screw 38 into engagement with the vertebra. Bone screw 28 is axially translated into opening 24. Head 30 engages slider 60 causing slider 60 to translate, in the direction shown by arrow A in FIG. 3, relative to wall 14 in plane P1. Slider 60 translates in plane P1 to engage bone screw 38. Slider 60 translates to an orientation, as shown in FIGS. 4 and 5, such that slider 60 overlaps opening 26 and engages shoulder 43 of bone screw 38 in a locking configuration to resist and/or prevent backout of bone screw 38 from opening 26.

Other components of system 10 are delivered to the surgical site, for example, the tether and the set screw. These components are disposed with bone screw 28, as described above, such that set screw is fixed with head 30, and the tether is fixed with bone screw 28 disposed along the vertebrae. Bone screw 28 is configured to support a tensile load with the tether over the selected section of vertebrae.

The components of system 10 are attached with a first side, such as, for example, a convex side of vertebrae to prevent growth of a selected section of vertebrae, while allowing for growth and adjustments to a second side, such as, for example, a concave side of vertebrae to provide treatment. Compression of vertebrae occurs along the convex side. As forces and/or force changes are applied to system 10, such as, for example, patient growth, trauma and degeneration, and/or system 10 component creep, deformation, damage and degeneration, the tether adapts with a responsive spring force to maintain the applied force transmitted from bone screw 28 substantially constant.

In one embodiment, system 10 includes an agent, which may be disposed, packed, layered and/or coated within, on or about the components and/or surfaces of system 10. In some embodiments, the agent may include bone growth promoting material, such as, for example, bone graft to enhance fixation of the bone fasteners and/or fixation elements with vertebrae.

In some embodiments, the agent may include therapeutic polynucleotides or polypeptides. In some embodiments, the agent may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as HA, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines.

The components of system 10 can be made of radiolucent materials such as polymers. Radiomarkers may be included for identification under x-ray, fluoroscopy, CT or other imaging techniques. In some embodiments, the agent may include one or a plurality of therapeutic agents and/or pharmacological agents for release, including sustained release, to treat, for example, pain, inflammation and degeneration.

In some embodiments, the use of microsurgical and image guided technologies may be employed to access, view and repair spinal deterioration or damage, with the aid of system 10. Upon completion of the procedure, the surgical instruments, assemblies and non-implant components of system 10 are removed from the surgical site and the incision is closed.

In some embodiments, the components of system 10 may be employed to treat progressive idiopathic scoliosis with or without sagittal deformity in either infantile or juvenile patients, including but not limited to prepubescent children, adolescents from 10-12 years old with continued growth potential, and/or older children whose growth spurt is late or who otherwise retain growth potential. In some embodiments, the components of system 10 and methods of use may be used to prevent or minimize curve progression in individuals of various ages.

In one embodiment, as shown in FIG. 6, system 10, similar to the systems and methods described with regard to FIGS. 1-5, includes a plate 112, similar to plate 12 described above. Plate 112 includes a wall 114, similar to wall 14 described, which extends between an end 116 and an end 118. Plate 112 has a double plate configuration such that wall 114 includes an opening 124 configured for disposal of bone screw 28, described above. Plate 112 includes an opening 126 disposed adjacent to opening 124 such that openings 124, 126 are disposed in communication. In one embodiment, openings 124, 126 partially overlap. Opening 126 is configured for disposal of bone screw 38, described above.

Plate 112 includes an opening 125 disposed at end 116 of plate 112. Opening 125 is spaced apart from openings 124, 126. Opening 125 is substantially circular and extends through the thickness of plate 112. Opening 125 is configured for disposal of a tool or instrument (not shown) such that the tool manipulates plate 112 to introduce and/or deliver plate 112 to a surgical site.

In operation, bone screw 38 is axially translated into opening 126 by inserting a driving tool (not shown) configured to engage a correspondingly shaped socket cavity 46 and rotating bone screw 38 into engagement with vertebral tissue. After bone screw 38 has been inserted within opening 126, bone screw 28 is axially translated into opening 124. As bone screw 28 is inserted within opening 124, head 30 of bone screw 28 engages head 40 and/or shoulder 43 of bone screw 38 to overlap head 40 and/or shoulder 43 to resist and/or prevent backout of bone screw 38 from opening 126.

In one embodiment, as shown in FIGS. 7-9, system 10, similar to the systems and methods described with reference to FIGS. 1-5, includes a plate 212, similar to plate 12 described above. Plate 212 includes a wall 214, similar to wall 14 described, which extends between an end 216 and an end 218 and defines a plane P2. Plate 212 has a surface 220 and a surface 222 oriented in a posterior and/or postero-lateral direction for engagement with vertebral tissue. Wall 214 includes an opening 224 configured for disposal of bone screw 28, described above. Wall 214 includes an opening 226 spaced apart from opening 224 along surface 220. Opening 226 is configured for disposal of bone screw 38, described above.

Plate 212 includes an inner surface 248 that defines a passageway 250. Passageway 250 is disposed between openings 224, 226. Inner surface 248 includes an extension, such as, for example, a ledge 252. Ledge 252 extends from inner surface 248 into passageway 250 and has a thickness t2. Ledge 252 defines a channel 254 of passageway 250. Channel 254 is disposed between openings 224, 226. Channel 254 defines a keyway for mating engagement with a key of a rotatable part, described below. In some embodiments, passageway 250 and/or channel 254 can be variously configured, such as, for example, those alternatives described herein.

System 10 includes a part, such as, for example, a slider 260 configured for movable disposal with passageway 250. Slider 260 includes a first portion, such as, for example, a lock 262, an intermediate portion 272, and a second portion, such as, for example, a key 274.

Lock 262 is configured for engagement with bone screws 28, 38, similar to portion 76, described above. Lock 262 has a substantially triangular configuration. In some embodiments, lock 262 can be variously configured, such as, for example, those alternatives described herein. Lock 262 includes an inclined surface 264 engageable with bone screw 28 to rotate slider 260 relative to wall 214 in plane P2.

Lock 262 includes an inclined surface 266 engageable with bone screw 38 and disposed at a perpendicular orientation relative to surface 264. In some embodiments, surface 266 can be disposed at other angular orientations such as acute or obtuse, co-axial and/or may be offset or staggered relative to surface 264. Lock 262 includes a surface 268 disposed between surfaces 264, 266 and having an arcuate configuration. Lock 262 includes a substantially planar underside surface 270 oriented in a direction facing surface 220 of plate 212 and configured to overlap with shoulder 43 of bone screw 38 in a locking orientation of plate 212. In some embodiments, surface 270 may be variously configured, such as, for example, those alternatives described herein. In some embodiments, surfaces 264, 266, 268 and/or 270 may have various surface configurations, such as, for example, those alternatives described herein.

Intermediate portion 272 extends between lock 262 and key 274. Intermediate portion 272 has a substantially arcuate cross section configuration and is configured for rotation in passageway 250. In some embodiments, the cross section of intermediate portion 272 can be variously configured, such as, for example, those alternatives described herein.

Key 274 extends from intermediate portion 272 and is configured for mating engagement with channel 254. Key 274 has a male configuration for corresponding engagement with the female configuration of the keyway of channel 254. Key 274 is oriented into alignment with the keyway of channel 254 such that key 274 passes through passageway 250 for assembly of slider 260 with wall 214. Key 274 is fully disposed with passageway 250 such that surface 270 engages surface 220. Key 274 is rotated in a clockwise or a counter-clockwise direction to dispose key 274 out of alignment with the keyway of channel 254. In this configuration, key 274 is oriented to engage ledge 252 to prevent disassembly of slider 260 from wall 214. As such, slider 260 is rotatable relative to inner surface 248 in plane P2. In some embodiments, key 274 is rotated 90 degrees out of alignment with the keyway of channel 254. In some embodiments, key 274 is rotatable through an angular range of 0 to 180 degrees while being disposed out of alignment with the keyway of channel 254 to prevent disassembly of slider 260 from wall 214.

In operation, plate 212 includes slider 260, which is oriented in an orientation such that such that key 274 is disposed within the keyway of channel 254 and slider 260 is keyed and retained with passageway 250, as shown in FIG. 8. Surface 270 of lock 262 is substantially flush with surface 220 of wall 214. Lock 262 is oriented in an orientation to overlap a portion of opening 224 such that bone screw 38 is moveable within opening 226. Bone screw 38 is axially translated into opening 226 and a drive tool is disposed with cavity 46 to rotate bone screw 38 into engagement with vertebral tissue.

Bone screw 28 is axially translated into opening 224. Head 30 of bone screw 28 engages surface 264 causing slider 260 to rotate, in the direction shown by arrow B in FIGS. 8 and 9, relative to inner surface 248 in plane P2. Slider 260 rotates in plane P2 such that surface 266 engages bone screw 38. Slider 260 rotates for translation in plane P2 to an orientation, as shown in FIG. 8, such that surface 270 of slider 260 overlaps a portion of opening 26 and engages shoulder 43 of bone screw 38. In this orientation, slider 260 engages shoulder 43 in a locking configuration of plate 212 with bone screw 38 to resist and/or prevent backout of bone screw 38 from opening 226.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal implant comprising:
a wall defining a plane and having a first surface and a second surface configured to engage tissue, the wall defining a first opening configured for disposal of a first bone fastener and a second opening configured for disposal of a second bone fastener, the wall including an inner surface that defines a passageway disposed between the first opening and the second opening; and
a part configured for disposal in the passageway, the first bone fastener being engageable with the part such that the part translates relative to the inner surface in the plane to engage the second bone fastener and to resist backout of the second bone fastener from the second opening.

2. A spinal implant as recited in claim 1, wherein the part is translatable along the passageway between a first orientation to overlap at least a portion of the first opening such that the second bone fastener is moveable within the second opening and a second orientation to overlap at least a portion of the second opening such that the part resists backout of the second bone fastener from the second opening.

3. A spinal implant as recited in claim 1, wherein the inner surface includes at least one flange that defines a channel of the passageway, at least a portion of the part being disposable in the channel.

4. A spinal implant as recited in claim 1, wherein the inner surface includes opposing flanges that each define a channel of the passageway, at least a portion of the part being disposable in each of the channels.

5. A spinal implant as recited in claim 1, wherein the inner surface includes at least one flange that defines a channel of the passageway, the part including at least one flange configured for disposal in the at least one channel.

6. A spinal implant as recited in claim 1, wherein the part includes an inclined surface engageable with the first bone fastener such that the part translates relative to the inner surface in the plane.

7. A spinal implant as recited in claim 1, wherein the part extends between a first end and a second end, the first end including an inclined cam surface and a lateral arcuate surface engageable with the first bone fastener such that the part translates relative to the inner surface in the plane.

8. A spinal implant as recited in claim 1, wherein the part extends between a first end and a second end, each end including an inclined cam surface and a lateral arcuate surface.

9. A spinal implant as recited in claim 1, wherein the part is rotatable in the passageway between a first orientation to overlap at least a portion of the first opening such that the second bone fastener is moveable within the second opening and a second orientation to overlap at least a portion of the second opening such that the part resists backout of the second bone fastener from the second opening.

10. A spinal implant as recited in claim 1, wherein the part includes a first portion configured to engage the bone fasteners, an intermediate portion and a second portion configured for mating engagement with the wall.

11. A spinal implant as recited in claim 10, wherein the second portion of the part has a male configuration and the inner surface of the wall has a female configuration for mating engagement.

12. A spinal implant as recited in claim 10, wherein the second portion of the part includes a key and the inner surface of the wall defines a keyway that mates with the second portion.

13. A spinal implant as recited in claim 10, wherein the first portion includes a first inclined surface engageable with the first bone fastener to rotate the part relative to the inner surface in the plane.

14. A spinal implant as recited in claim 13, wherein the first portion includes a second inclined surface engageable with the second bone fastener and disposed at angular orientation relative to the first inclined surface.

15. A spinal implant as recited in claim 13, wherein the first portion includes a second inclined surface engageable with the second bone fastener and disposed at a perpendicular orientation relative to the first inclined surface within the plane.

16. A spinal implant as recited in claim 1, wherein the part includes a sliding lock.

17. A spinal implant as recited in claim 1, wherein the second surface includes at least one transverse extension configured to penetrate tissue.

18. A spinal implant comprising:
a plate defining a plane and having a first surface and a second surface configured to engage vertebral tissue, the plate defining a first opening configured for disposal of a first bone fastener having a proximal portion that defines an implant cavity and a distal portion configured to penetrate the vertebral tissue,
the plate defining a second opening configured for disposal of a second bone fastener having a proximal portion that includes a shoulder and a distal portion configured to penetrate the vertebral tissue,
the plate including an inner surface having flanges that define a channel disposed between the first opening and the second opening; and
a slider,
wherein the slider is translatable along the channel in the plane between a first orientation such that the second bone fastener is moveable within the second opening and a second orientation such that the first bone fastener engages the slider to translate the slider relative to the inner surface to overlap the shoulder to resist backout of the second bone fastener from the second opening.

19. A spinal implant comprising:
a plate defining a plane and having a first surface and a second surface configured to engage vertebral tissue, the plate defining a first opening configured for disposal of a first bone fastener having a proximal portion that defines an implant cavity and a distal portion configured to penetrate the vertebral tissue,
the plate defining a second opening configured for disposal of a second bone fastener having a proximal portion that includes a shoulder and a distal portion configured to penetrate the vertebral tissue,
the plate including an inner surface that defines a first mating part disposed between the first opening and the second opening; and
a rotatable part including a slider configured to engage the bone fasteners, an intermediate portion and a second mating part configured for mating engagement with the first mating part,
wherein the rotatable part is movable in the plane between a first orientation such that the second bone fastener is moveable within the second opening and a second orientation such that the first bone fastener engages the slider to rotate the slider relative to the inner surface to overlap the shoulder to resist backout of the second bone fastener from the second opening.

20. A spinal implant as recited in claim 19, wherein the first portion includes a first inclined surface engageable with the first bone fastener to rotate the part relative to the inner surface in the plane and a second inclined surface disposed at an angular orientation relative to the first inclined surface.
